(19) United States
(12) Patent Application Publication
(10) Pub. No.: US 2023/0310014 A1
(43) Pub. Date: Oct. 5, 2023

(54) CATHETER ASSEMBLY AND METHOD OF USE

(71) Applicant: TERUMO KABUSHIKI KAISHA, Tokyo (JP)

(72) Inventors: Futoshi TOMITA, Obu-shi (JP); Tetsuya FUKUOKA, Hadano-shi (JP)

(73) Assignee: TERUMO KABUSHIKI KAISHA, Tokyo (JP)

(21) Appl. No.: 18/190,628

(22) Filed: Mar. 27, 2023

(30) Foreign Application Priority Data
Apr. 1, 2022 (JP) 2022-062076

(51) Int. Cl. A61B 17/22; A61M 25/00

(52) U.S. Cl. CPC A61B 17/22; A61M 25/0097; A61M 25/01

(57) ABSTRACT

A catheter assembly in which a microcatheter of a rapid exchange type is a first catheter and is inserted into a lumen of a guiding catheter that is a second catheter. The first and second catheters are configured to make a clearance slightly exceeding 0.1 mm to reduce a step, so that a distal end opening of the guiding catheter is prevented from rubbing against an inner surface of a blood vessel in a way that would otherwise rupture plaque and scatter cholesterol crystals.

### CROSS-REFERENCES TO RELATED APPLICATIONS

This application is based on and claims priority to Japanese Patent Application No. 2022-62076 filed on Apr. 1, 2022, the entire content of which is incorporated herein by reference.

### TECHNOLOGICAL FIELD

The present invention generally relates to a catheter assembly inserted into the heart or tissue around the heart, especially the left and right coronary arteries (hereinafter also simply referred to as “the coronary arteries”) to be used, and a method of use. More specifically, the present invention relates to a catheter assembly and a method of use for reducing cholesterol crystal embolism, which might rarely occur when a guiding catheter for introducing a treatment catheter especially such as a dilation catheter for PTCA or a stent delivery catheter into a target site of a blood vessel is advanced into the blood vessel.

### BACKGROUND DISCUSSION

Cholesterol, which is a fat-soluble compound dissociated from low-density lipoprotein (LDL) and crystallized, may be accumulated in intravascular atheroma or blood vessel wall plaque caused by arteriosclerosis.

Cholesterol crystal embolism (CCE) refers to occlusion or an inflammatory response occurring in a fine blood vessel such as a peripheral blood vessel when plaque in a blood vessel is ruptured due to catheter treatment, thrombolytic therapy or the like and needle-shaped cholesterol crystals having a diameter of 55 to 200 μm diffuse into a body as disclosed in Hase, The Journal of the Japanese Society of Internal Medicine, 105(5) 2016: 850-856.

Specifically, it is said that BlueToe syndrome due to occlusion of the peripheral artery by CCE (in Hase, The Journal of the Japanese Society of Internal Medicine, 105(5) 2016: 850-856), and acute renal failure that occludes glomeruli (in Vuurmans, Heart, 2010; 96: 1538-1542) is less likely to occur in transradial artery intervention (TRI) than in transfemoral intervention (TFI) in percutaneous coronary intervention (PCI).

### SUMMARY

JP 2020-151465 A discloses a method for sucking and removing cholesterol crystals and the like at a lesion, but it is not intended to suppress scattering of cholesterol crystals generated when the guiding catheter is advanced to the lesion. Takahashi et al., The American Journal of Cardiology, Jan. 2, 2022 (online ahead of print) discloses that a catheter referred to as an over-the-wire type inner sheath is inserted into a guiding catheter, and U.S. Pat. No. 10,322,260 and U.S. Patent Application Publication No. 2007/0149927 disclose a catheter assembly of a guiding catheter inserted from the radial artery and an inner sheath; a clearance being a difference between an inner diameter of a guiding catheter distal end opening and an outer diameter of the inner sheath is 0.1 mm, and in a case where the guiding catheter and the inner sheath are extremely long to be longer than 2 m, friction might increase.

Furthermore, since there is a case where an insertion assisting tool of a rapid exchange type as in U.S. Patent Application Publication No. 2009/0264865 has no clearance between the tool and an inner diameter of a guiding catheter or a clearance of less than 0.1 mm, there is a possibility that the tool does not protrude from a guiding catheter distal end opening.

(1) A catheter assembly disclosed here includes a first catheter comprised of a first shaft that includes a distal end and a proximal end at opposite axial ends of the first shaft, with the first shaft including a first distal end opening at the distal end of the first shaft and a first proximal end opening at the proximal end of the first shaft, and the first catheter including a first lumen extending between the first distal end opening and the first proximal end opening. The catheter assembly also includes a second catheter comprised of a second shaft that includes a distal end and a proximal end at opposite axial ends of the second shaft, with the second shaft including a second distal end opening at the distal end of the second shaft and a second proximal end opening at the proximal end of the second shaft, and the second catheter including a second lumen extending between the second distal end opening and the second proximal end opening, wherein the second shaft is longer than the first shaft, and the second catheter includes a second hub at the proximal end of the second shaft, and the second catheter having both an outer diameter and an inner diameter at the second distal end opening. The first catheter is positionable in the second lumen of the second shaft and is axially positionable relative to the second shaft such that a distal portion of the first shaft extends distally beyond the distal end of the second shaft. A clearance between an inner surface of the second shaft and an outer surface of the first shaft at the second distal end opening when the first catheter is positioned in the second lumen of the second shaft and the distal portion of the first shaft extends distally beyond the distal end of the second shaft exceeds 0.1 mm.

(2) The catheter assembly according to (1) described above, including a push rod on a proximal end side of the first shaft.

In accordance with another aspect of the disclosure here, a catheter assembly comprises a first catheter and a second catheter. The first catheter is comprised of a tubular first shaft and a rod fixed to the tubular first shaft, the first shaft including a distal end and a proximal end at opposite axial ends of the first shaft, the first shaft including a first distal end opening at the distal end of the first shaft and a first proximal end opening at the proximal end of the first shaft, the first catheter including a first lumen extending throughout an axial extent of the first shaft from the first distal end opening to the first proximal end opening, the rod extending away from the proximal end of the first shaft, the first shaft including a distal portion that is a tapering part that tapers so that an outer diameter of the first shaft portion at a proximal end of the tapering part is greater than the outer diameter of the first shaft portion at a distal end of the tapering part, the first shaft possessing an axial length from the distal end of the first shaft to the proximal end of the first shaft. The second catheter comprises a second shaft that includes a distal end and a proximal end at opposite axial ends of the second shaft, the second shaft including a second distal end opening at the distal end of the second shaft and a second proximal end opening at the proximal end of the second shaft, the second catheter including a second lumen extending through an axial extent of the second shaft from the second distal end opening to the second proximal end opening, the second shaft possessing an axial length from the distal end of the second shaft to the proximal end of the second shaft, the second catheter including a second hub at the proximal end of the second shaft, the second catheter having both an outer diameter and an inner diameter at the second distal end opening. The axial length of the second catheter is greater than the axial length of the first catheter. The first catheter is positionable in the second lumen of the second shaft and is axially positionable relative to the second shaft such that the tapering part of the first shaft is positioned distally beyond the distal end of the second shaft. The clearance between an inner surface of the second shaft and an outer surface of the first shaft at the distal end of the second shaft when the first catheter is positioned in the second lumen of the second shaft and the tapering part of the first shaft is positioned distally beyond the distal end of the second shaft exceeds 0.1 mm.

(3) Another aspect of the disclosure involves a method of use of a catheter for reducing cholesterol crystal embolism occurring during catheter treatment. The method comprises positioning the catheter assembly for insertion into a first blood vessel, wherein the catheter assembly comprises: a first catheter comprised of a first shaft that includes a first distal end opening, a first proximal end opening and a first lumen extending between the first distal end opening and the first proximal end opening; a second catheter comprised of a second shaft that includes a second distal end opening, a second proximal end opening and a second lumen extending between the second distal end opening and the second proximal end opening, the second shaft being longer than the first shaft, the second catheter including a hub at a proximal end of the second shaft, the second catheter having both an outer diameter and an inner diameter at the second distal end opening; the first catheter being positioned in the second lumen of the second shaft; a guide wire located in the first lumen in the first shaft; and a clearance between an inner surface of the second shaft and an outer surface of the first shaft at the second distal end opening exceeding 0.1 mm. The method further comprises inserting the catheter assembly into the first blood vessel, advancing the catheter assembly along the first blood vessel while a distal portion of the first shaft extends distally beyond the distal end of the second shaft and while the guide wire is located in the first lumen in the first shaft and extends distally beyond the distal end of the first shaft, and advancing the catheter assembly from the first blood vessel to a second blood vessel with a branch and advancing at least the second catheter to a third blood vessel having a lesion on which catheter treatment is to be performed.

According to the disclosure here, to eliminate a large step, which is a clearance of about 1 mm between a distal end opening of a guiding catheter and a guide wire inserted thereinto, a catheter assembly in which a microcatheter of a rapid exchange type is inserted into a lumen of the guiding catheter is used to make the clearance slightly exceed 0.1 mm to reduce the step, so that the distal end opening of the guiding catheter is prevented from rubbing an inner surface of a blood vessel to rupture plaque and scatter cholesterol crystals. In the absence of the microcatheter, a relatively large clearance would exist between the inner surface of the guiding catheter and the outer surface of the guide wire, and so the guiding catheter would be able to move laterally a relatively large amount, meaning the guiding catheter may rub against the inner surface of the blood vessel. Positioning the catheter (microcatheter) inside the guiding catheter reduces the clearance between the inner surface of the guiding catheter and the outer surface of the microcatheter and so the guiding catheter is not able to move laterally to the same extent, thus reducing the likelihood that the guiding catheter will rub against the inner surface of the blood vessel. If the clearance becomes too small, frictional resistance between the inner surface of the guiding catheter and the outer surface of the microcatheter may undesirably increase. The catheter assembly thus seeks to identify a clearance that will both lessen the possibility that the guiding catheter will rub against the inner surface of the blood vessel and reduce frictional resistance between the inner surface of the guiding catheter and the outer surface of the microcatheter.

### BRIEF DESCRIPTION OF THE DRAWINGS

FIG. 1 is a cross-sectional view in an axial direction of a catheter assembly according to an embodiment.

FIG. 2 is an enlarged view of a distal end of the catheter assembly according to the embodiment.

FIGS. 3A, 3B, and 3C are cross-sectional views taken along the section lines 3A-3A, 3B-3B, and 3C-3C, respectively, of the catheter assembly according to the embodiment.

FIG. 4 is an enlarged cross-sectional view of the catheter assembly shown in FIG. 3B, illustrating dimensions of the catheter assembly.

### DETAILED DESCRIPTION

Here, a first catheter refers to, for example, a microcatheter 10, and the microcatheter 10 includes a first shaft 11, which is a microcatheter shaft, a first distal end opening 18, which is a microcatheter distal end opening of the microcatheter shaft 11, a first proximal end opening 19, which is a microcatheter proximal end opening, and a first lumen 15, which is a microcatheter lumen extending between the first distal end opening 18 and the first proximal end opening 19.

The first lumen 15 of the first shaft 11 is intended to pass only a guide wire 30, and a therapeutic catheter such as a general balloon catheter or a stent delivery catheter does not pass therethrough; however, a therapeutic catheter such as a balloon catheter or a stent delivery catheter having a reduced outer diameter may pass through the first lumen 15.

The first shaft 11 may have a tapered distal end, or may have a reinforcing body (not illustrated) embedded therein; the reinforcing body may be embedded at least in a first shaft base 12 at a position on a distal end side from the first proximal end opening 19, and the reinforcing body may be formed by braiding or coil-winding a metal wire such as a stainless alloy, a nickel-titanium alloy, or tungsten.

Here, a second catheter refers to a guiding catheter 20 including a distal tip for introducing a treatment catheter especially such as a dilation catheter for PTCA and a stent delivery catheter to a target site of a blood vessel, the guiding catheter 20 includes a distal tip 21, a second shaft 22, which is a guiding catheter shaft, a second distal end opening 25, which is a guiding catheter distal end opening, a second proximal end opening 26, which is a guiding catheter proximal end opening, and a second lumen 23, which is a guiding catheter lumen extending between the second distal end opening 25 and the second proximal end opening 26, and a guiding catheter hub (second hub) 24, communicating with the second distal end opening 25 and the second proximal end opening 26, is attached to a proximal end of the second shaft 22.

The second shaft 22 may further include the distal tip 21 at a distal end thereof. The second shaft 22 may be obtained or formed by winding a reinforcing body around a PTFE inner layer, and further providing an outer layer outside of the reinforcing body so that the wound reinforcing body is embedded. The reinforcing body may be embedded in the second shaft 22 at least from a position closer to the proximal end than the distal tip 21 to the second proximal end opening 26. The reinforcing body may be formed by braiding or coil-winding a metal wire such as a stainless alloy, a nickel-titanium alloy, or tungsten. A hydrophilic lubricating coat layer may be provided on a part of an outer surface of each of the distal tip 21 and the second shaft 22.

The second catheter 20 may have a distal shape having a curved or bent portion at the distal end thereof, such as a Judkins shape, but such a distal shape is not necessarily required.

A second shaft length L2, which is a second catheter shaft length, is only required to be longer than a first shaft length L1, which is a shaft length of the first catheter, and the first shaft length L1 may be, for example, a length from the first distal end opening 18 to a first shaft proximal end or a length from the first distal end opening 18 to the first proximal end opening 19.

The first shaft 11 may include a push rod 16 fixed to the first shaft 11 and extending from the proximal end of the first shaft, and a first hub 17, which is a microcatheter tab, on a proximal end of the push rod 16.

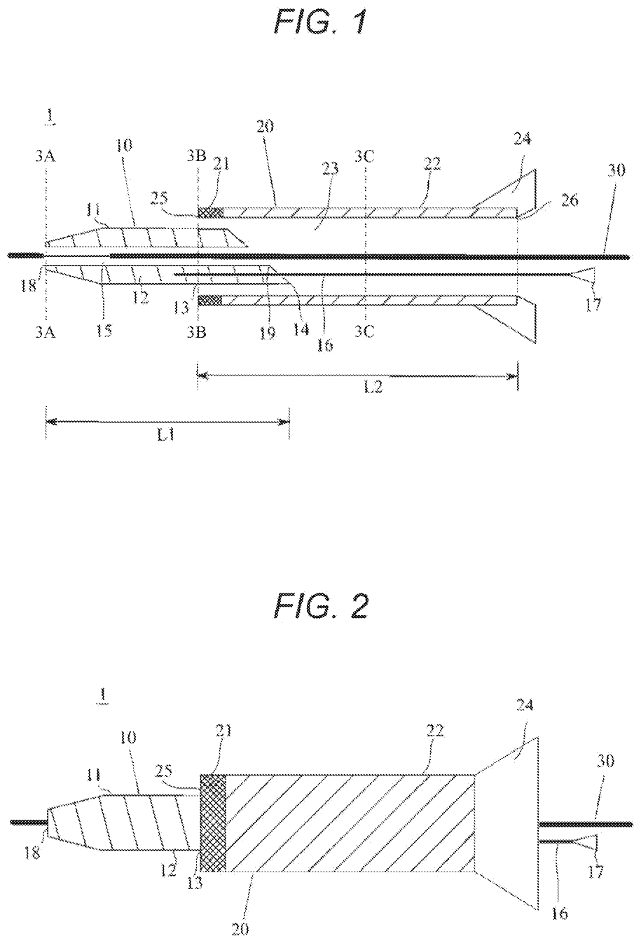

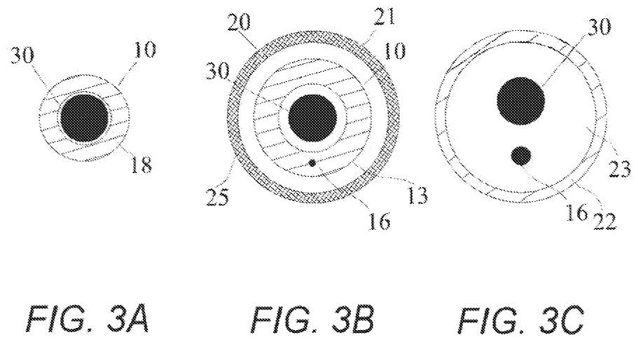

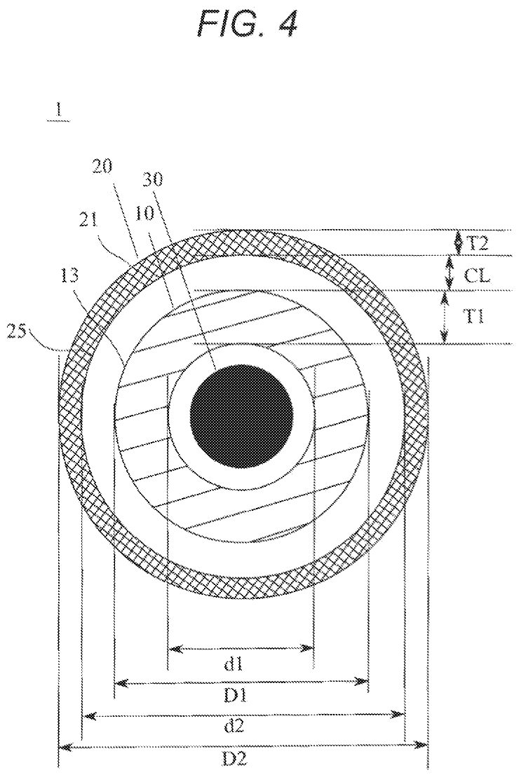

The push rod 16 may be a wire having a plate shape with a circular, or at least partially arc-shaped or ribbon shaped cross section, or may be a half pipe (a wire with a semicircular cross-section); and the first hub 17 may be fixed by adhesion or the like, or may be a detachable type, or it is also possible that there is no first hub 17. It is only required that the distal end of the push rod 16 does not protrude to the distal end from the first distal end opening 18 of the first shaft 11. That is, the push rod 16 preferably does not protrude distally beyond the first distal end opening 18 of the first shaft 11. It is preferable that the distal end of the push rod 16 is present at a position protruding from the second distal end opening 25 when the first catheter advances to a predetermined position as illustrated in FIGS. 1 and 3B, because the distal shape of the second catheter 22 may be straightened. However, it is also possible that the distal end of the push rod 16 may be on the proximal end side from the second distal end opening 25. As shown in FIGS. 3A-3C, the push rod 16 may be a solid rod, and the push rod 16 and the first shaft 11 may be other than coaxial (i.e., the push rod 16 and the first shaft 11 may be non-coaxial).

The first shaft 11 may include the push rod 16 embedded in a multilayer tube including the reinforcing body or a single-layer tube including no reinforcing body, or may include the push rod 16 attached to an outer side of the first shaft 11 on the distal end side from a proximal end 14 of the first shaft 11. The first shaft 11 may include a physical property transition portion that becomes harder from the distal end toward the base, or may be provided with a hydrophilic lubricating coat at the distal end.

The first catheter 10 is inserted into the second lumen 23 of the second catheter 20 and advanced to a predetermined position to achieve a state in which the first shaft 11 of the first catheter 10 protrudes from the guiding catheter distal end opening 25. The predetermined position is not especially limited, but when the first catheter 10 advances to the predetermined position, the guide wire 30 and the first distal end opening 18, preferably the distal portion of the first shaft 11, protrude from (i.e., distally beyond) the second distal end opening 25 to the distal end side. A distal end protruding length of the first shaft is a distance between the first distal end opening 18 and the second distal end opening 25, which is 10 mm to 200 mm, preferably 30 mm to 150 mm.

A length from the second distal end opening 25 to the second proximal end opening 26 is defined as a second shaft length L2, an outer diameter of the second catheter distal end opening is defined as a second outer diameter D2, an inner diameter of the second catheter distal end opening is defined as a second inner diameter d2, an outer diameter of the first shaft at a position of the second catheter distal end opening is defined as a first outer diameter D1, an inner diameter thereof is defined as a first inner diameter d1, a first wall thickness is defined as T1, and a difference between the second inner diameter d2 and the first outer diameter D1 is defined as a clearance CL.

Although the first wall thickness T1 is defined as (D1−d1)/2 and a second wall thickness T2 is defined as (D2−d2)/2, an average value of measured wall thicknesses may be used.

Note that, each dimension may be constant from the distal end to the proximal end, and D2 may be smaller than an outer diameter of the second proximal end opening 26, or d2 may be smaller than an inner diameter of the proximal end opening 26.

As for the clearance CL, in order to eliminate a large step, which is a clearance of about 1 mm between the second distal end opening 25 of the second catheter 20 and the guide wire 30 inserted thereinto, a catheter assembly 1 obtained by inserting or positioning the first catheter 10 of a rapid exchange type into the second lumen 23 of the second catheter 20 is used to make the clearance slightly exceed 0.1 mm to reduce the step, so that the second distal end opening 25 or the distal tip 21 is prevented from rubbing an inner surface of the blood vessel to rupture plaque and scatter a cholesterol crystal.

The clearance CL is preferably more than 0.1 mm and 0.5 mm or less, and more preferably 0.15 mm to 0.45 mm. As a result, frictional resistance between an inner surface of the second lumen 23 and an outer surface of the first shaft 11 can be reduced.

As the guide wire 30, an angiographic guide wire having an outer diameter of 0.89 mm (0.035 inch) referred to as an angiographic guide wire (for example, a Radifocus Guide Wire M® (registered trademark) manufactured by Terumo Corporation, hereinafter, simply referred to as a guide wire) is mainly used, but a therapeutic guide wire used for PTCA or the like referred to as a PTCA guide wire (for example, a Terumo PTCA guide wire, hereinafter referred to as a therapeutic guide wire) having an outer diameter of 0.36 mm (0.014 inch) may also be used; the outer diameter or use thereof is not especially limited.

Hereinafter, an operation (procedure) when engaging with the left coronary artery ostium having a lesion using the guiding catheter 20, which is the second catheter, and the catheter assembly 1 is described in detail. These operations are performed while checking a position and a posture of the catheter assembly 1 under X-ray fluoroscopy.

Here, the right radial artery into which the catheter assembly 1 is inserted is a first blood vessel, the aorta is a second blood vessel with a branch, and the left coronary artery is a third blood vessel (branch of the second blood vessel) having a lesion on which catheter treatment is performed.

The right radial artery is punctured with a catheter introducer, and the catheter assembly 1 is inserted into the catheter introducer in a state in which the guide wire 30 having an outer diameter of 0.89 mm (0.035 inch) is inserted into (positioned in) the first lumen 15 of the microcatheter 10, which is the first catheter of the catheter assembly 1, as illustrated in FIGS. 1 to 3. In a state in which both the guide wire 30 and the microcatheter 10 protrude from the second distal end opening 25 of the guiding catheter 20, a distal shape of the guiding catheter 10 is substantially linear due to rigidity of the guide wire 30.

After the catheter assembly 1 is inserted into the right radial artery from the catheter introducer in this manner, a distal end of the catheter assembly is advanced from the brachiocephalic artery to the ascending aorta with the guide wire preceding the same. When the distal tip 21 is located about 10 cm above the left coronary artery ostium of the left coronary artery or exceeds the aortic arch, the advance of the catheter assembly 1 is stopped, and the microcatheter 10 and the guide wire 30 are removed to make the distal shape of the guiding catheter 20 its original curved shape (natural state). That is, after removing the microcatheter 10 and the guide wire 30 from the guiding catheter 10, the distal shape or distal portion of the guiding catheter 20 assumes its original curved shape.

Next, when the guiding catheter 20 is slowly pushed forward while checking a distal end position of the guiding catheter 20 or the position of the distal tip 21, the distal tip 21 of the guiding catheter 20 moves downward while being in contact with a left inner wall of the ascending aorta and is inserted into the left coronary artery ostium. The distal shape at that time is a shape that facilitates engagement.

In a case where the distal end of the guiding catheter 20 is directed in a direction different from the left coronary artery ostium, the guiding catheter 20 is slightly rotated in a counterclockwise direction to direct the distal end in the direction of the left coronary artery ostium, and is slowly pushed forward in this direction. As a result, the distal end opening is easily inserted into the left coronary artery ostium and engaged in this state.

Next, a connector of a contrast agent injection instrument is connected to the second hub 24 including a Luer taper of the guiding catheter 20, and a contrast agent is injected. The injected contrast agent passes through the second lumen 23 via the second hub 24, and is ejected from the second distal end opening 25 into the left coronary artery. As a result, a stenosed part (diseased part) in the left coronary artery is imaged and its position is identified, but this operation is not essential.

Next, the connector of the contrast agent injection instrument is removed from (disconnected from) the second hub 24. Thereafter, a treatment catheter such as a balloon catheter for PTCA is inserted into the second lumen 23 from the second hub 24 together with a new therapeutic guide wire having an outer diameter of 0.36 mm (0.014 inch) to reach the stenosed part as a target site and treatment is performed thereon.

Specifically, a balloon portion of the balloon catheter is pushed forward to the stenosed part with the therapeutic guide wire preceding the same, and the balloon is dilated or expanded to perform a dilation treatment of the stenosed part.

The guiding catheter 20 may be engaged with the coronary artery using the catheter assembly 1 disclosed here via the left radial artery, or may be inserted from the femoral artery via the abdominal aorta; in these cases, the procedure similar to that described above can be performed.

In the above description, the case where the balloon catheter for PTCA is used as the treatment catheter is described, but the treatment catheter is not limited thereto; and even in a case where a stent delivery catheter that delivers a stent to release at a stenosed part is used, for example, the procedure is substantially similar.

Also in a case where there is a stenosed part in the right coronary artery and this is subjected to dilation treatment, the procedure similar to that described above can be performed according to this disclosure.

TABLE 1

| | First catheter | | Second catheter | | | | | Comparison | |
|---|---|---|---|---|---|---|---|---|---|
| | D1 (mm) | d1 (mm) | D2 (mm) | d2 (mm) | T1 (mm) | CL (mm) | T2 (mm) | result | Application |
| Example 1 | 1.90 | 1.15 | 2.66 | 2.16 | 0.375 | 0.260 | 0.250 | T2 < CL < T1 | Femoral |
| Example 2 | 2.09 | 1.15 | 2.66 | 2.29 | 0.470 | 0.200 | 0.185 | T2 < CL < T1 | Femoral |
| Example 3 | 2.00 | 1.10 | 2.43 | 2.20 | 0.450 | 0.200 | 0.115 | T2 < CL < T1 | Radial/Femoral |
| Example 4 | 1.68 | 1.05 | 2.36 | 2.06 | 0.315 | 0.380 | 0.150 | T2 < T1 < CL | Radial/Femoral |
| Example 5 | 1.68 | 1.05 | 2.40 | 2.06 | 0.315 | 0.380 | 0.170 | T2 < T1 < CL | Radial/Femoral |
| Example 6 | 1.35 | 1.05 | 2.06 | 1.80 | 0.150 | 0.450 | 0.130 | T2 < T1 < CL | Radial/Femoral |
| Example 7 | 1.35 | 1.05 | 2.09 | 1.80 | 0.150 | 0.450 | 0.145 | T2 < T1 < CL | Radial/Femoral |
| Example 8 | 1.96 | 1.15 | 2.66 | 2.16 | 0.405 | 0.200 | 0.250 | CL < T2 < T1 | Femoral |
| Example 9 | 2.11 | 1.15 | 2.66 | 2.29 | 0.480 | 0.180 | 0.185 | CL < T2 < T1 | Femoral |
| Example 10 | 2.09 | 1.15 | 2.43 | 2.20 | 0.470 | 0.110 | 0.115 | CL < T2 < T1 | Femoral |
| Example 11 | 1.74 | 1.10 | 2.10 | 1.85 | 0.320 | 0.110 | 0.125 | CL < T2 < T1 | Femoral |
| Comparative | 1.78 | 1.15 | 2.06 | 1.85 | 0.315 | 0.070 | 0.105 | CL < T2 < T1 | Radial OTW |
| Example 1 | | | | | | | | | |

The guiding catheter 20 having an outer diameter of 2.33 mm (7 Fr), 2.66 mm (8 Fr) or more as in Examples 1 to 3 may have a combination satisfying T2<CL<T1 when CL is more than 0.1 mm. An increase in friction between the inner surface of the second lumen 23 of the guiding catheter 20 and the outer surface of the first shaft 11 is prevented. It is possible to reduce CCE said to be more likely to occur especially when used in a Femoral approach than in a Radial approach.

Alternatively, in the guiding catheter having an outer diameter of 2.0 mm (6 Fr) or more in which both the Radial approach and the Femoral approach may be used as in Examples 4 to 7, a combination satisfying T2<T1<CL is possible when CL is more than 0.1 mm. An increase in friction between the inner surface of the second lumen 23 of the guiding catheter 20 and the outer surface of the first shaft 11 is prevented, and CCE may be reduced also in the Radial approach.

Furthermore, although this is used in the Femoral approach in Examples 8 to 11, since the microcatheter 10 is of the rapid exchange type, a portion where the outer surface of the first shaft 11 and the inner surface of the second lumen 23 are in contact with each other is short, so that the clearance CL is only required to be more than 0.1 mm; with an existing over-the-wire type inner sheath as in Comparative Example 1, the clearance CL is too small, so that a frictional resistance might be large.

In a case where the distal tip 21 and the second shaft mainly used in the Radial approach are thin, specifically, with the guiding catheter 20 having small T2, if CL exceeds 0.1 mm, it is possible to prevent the thin second distal end opening 25 or the distal tip 21 from scraping the plaque on the blood vessel surface and to reduce the outer diameter of the guiding catheter 20 as CL<T2<T1.

Note that, in a case where a body size is large and the blood vessel of the radial artery is thick, the guiding catheter assembly 1 in which the outer diameter of the guiding catheter 20 is 8 Fr may be used in the Radial approach as long as this may be safely inserted into the blood vessel.

The inner diameter and the outer diameter of the distal end opening and the shaft may be constant, or may decrease toward the distal end.

An application of the catheter assembly 1 disclosed as an example of the new catheter assembly disclosed here is not especially limited, and can be applied to, for example, the guiding catheter 20 for introducing an atherectomy catheter, an ultrasonic imaging catheter or the like, in addition to the above-described catheter. An introduced site in the living body is not limited to the radial artery but may be the femoral artery, and it goes without saying that a lesion to be treated is not limited to the coronary artery but may be lower extremity arteries, blood vessels connected to organs such as the hepatic artery, the prostatic artery, or the uterine artery, blood vessels connected to cerebrovascular vessels such as the carotid artery or the vertebral artery, the cerebrovascular vessels or the like.

Alternatively, the microcatheter 10 may be packed in the guiding catheter 20, or a single guiding catheter 20 and a single microcatheter 10 sterilized in a packaging material may be assembled at the time of catheter treatment and used as the catheter assembly 1, or may be packaged in an assembled state and sterilized so as to be immediately taken out and used in the operating room.

The detailed description above describes embodiments of a catheter assembly and method of use representing examples of the new catheter assembly and method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A catheter assembly comprising:
a first catheter comprised of a first shaft that includes a distal end and a proximal end at opposite axial ends of the first shaft, the first shaft including a first distal end opening at the distal end of the first shaft and a first proximal end opening at the proximal end of the first shaft, the first catheter including a first lumen extending between the first distal end opening and the first proximal end opening;
a second catheter comprised of a second shaft that includes a distal end and a proximal end at opposite axial ends of the second shaft, the second shaft including a second distal end opening at the distal end of the second shaft and a second proximal end opening at the proximal end of the second shaft, the second catheter including a second lumen extending between the second distal end opening and the second proximal end opening, the second shaft being longer than the first shaft, the second catheter including a second hub at the proximal end of the second shaft and communicating with the second lumen, the second catheter having both an outer diameter and an inner diameter at the second distal end opening; the first catheter being positionable in the second lumen of the second shaft and being axially positionable relative to the second shaft such that a distal portion of the first shaft extends distally beyond the distal end of the second shaft; and
a clearance between an inner surface of the second shaft and an outer surface of the first shaft at the second distal end opening when the first catheter is positioned in the second lumen of the second shaft and the distal portion of the first shaft extends distally beyond the distal end of the second shaft exceeding 0.1 mm.

2. The catheter assembly according to claim 1, further comprising: a push rod on a proximal end side of the first shaft.

3. The catheter assembly according to claim 1, wherein the distal portion of the first includes a tapered part that extends to the distal end of the first shaft, the clearance exceeding 0.1 mm when an entirety of the tapered part of the first shaft is distal of the distal end of the second shaft.

4. The catheter assembly according to claim 1, wherein the first catheter further comprises a rod fixed to the first shaft and extending in a proximal direction away from the proximal end of the first shaft, the rod possessing a proximal end that is positioned proximal of the proximal end of the second shaft when the first catheter is positioned in the second lumen of the second shaft and the distal portion of the first shaft extends distally beyond the distal end of the second shaft.

5. The catheter assembly according to claim 4, wherein the first catheter possesses a wall thickness T1, the second catheter possesses a wall thickness T2, and a clearance CL exists between an inner surface of the second shaft and an outer surface of the first shaft, and wherein T2<CL<T1.

6. The catheter assembly according to claim 4, wherein the first catheter possesses a wall thickness T1, the second catheter possesses a wall thickness T2, and a clearance CL exists between an inner surface of the second shaft and an outer surface of the first shaft, and wherein T2<T1<CL.

7. A catheter assembly comprising:
a first catheter comprised of a tubular first shaft and a rod fixed to the tubular first shaft, the first shaft including a distal end and a proximal end at opposite axial ends of the first shaft, the first shaft including a first distal end opening at the distal end of the first shaft and a first proximal end opening at the proximal end of the first shaft, the first catheter including a first lumen extending throughout an axial extent of the first shaft from the first distal end opening to the first proximal end opening, the rod extending away from the proximal end of the first shaft, the first shaft including a distal portion that is a tapering part that tapers so that an outer diameter of the first shaft portion at a proximal end of the tapering part is greater than the outer diameter of the first shaft portion at a distal end of the tapering part, the first shaft possessing an axial length from the distal end of the first shaft to the proximal end of the first shaft;
a second catheter comprised of a second shaft that includes a distal end and a proximal end at opposite axial ends of the second shaft, the second shaft including a second distal end opening at the distal end of the second shaft and a second proximal end opening at the proximal end of the second shaft, the second catheter including a second lumen extending through an axial extent of the second shaft from the second distal end opening to the second proximal end opening, the second shaft possessing an axial length from the distal end of the second shaft to the proximal end of the second shaft, the second catheter including a second hub at the proximal end of the second shaft, the second catheter having both an outer diameter and an inner diameter at the second distal end opening;
the axial length of the second catheter being greater than the axial length of the first catheter;
the first catheter being positionable in the second lumen of the second shaft and being axially positionable relative to the second shaft such that the tapering part of the first shaft is positioned distally beyond the distal end of the second shaft; and
a clearance between an inner surface of the second shaft and an outer surface of the first shaft at the distal end of the second shaft when the first catheter is positioned in the second lumen of the second shaft and the tapering part of the first shaft is positioned distally beyond the distal end of the second shaft exceeding 0.1 mm.

8. The catheter assembly according to claim 7, wherein the rod possesses a proximal end that is positioned proximal of the proximal end of the second shaft when the first catheter is positioned in the second lumen of the second shaft and the tapering part of the first shaft is positioned distally beyond the distal end of the second shaft.

9. The catheter assembly according to claim 8, further comprising a hub fixed to the proximal end of the rod.

10. The catheter assembly according to claim 7, wherein the rod is a solid rod.

11. The catheter assembly according to claim 7, wherein the rod and the first shaft are other than coaxial.

12. The catheter assembly according to claim 7, wherein the first catheter possesses a wall thickness T1, the second catheter possesses a wall thickness T2, and a clearance CL exists between an inner surface of the second shaft and an outer surface of the first shaft, and wherein T2<CL<T1.

13. The catheter assembly according to claim 7, wherein the first catheter possesses a wall thickness T1, the second catheter possesses a wall thickness T2, and a clearance CL exists between an inner surface of the second shaft and an outer surface of the first shaft, and wherein T2<T1<CL.

14. A method of use of a catheter assembly for reducing cholesterol crystal embolism occurring during catheter treatment, the method comprising:
positioning the catheter assembly for insertion into a first blood vessel, the catheter assembly comprising: a first catheter comprised of a first shaft that includes a first distal end opening, a first proximal end opening and a first lumen extending between the first distal end opening and the first proximal end opening; a second catheter comprised of a second shaft that includes a second distal end opening, a second proximal end opening and a second lumen extending between the second distal end opening and the second proximal end opening, the second shaft being longer than the first shaft, the second catheter including a hub at a proximal end of the second shaft, the second catheter having both an outer diameter and an inner diameter at the second distal end opening; the first catheter being positioned in the second lumen of the second shaft; a guide wire located in the first lumen in the first shaft; and a clearance between an inner surface of the second shaft and an outer surface of the first shaft at the second distal end opening exceeding 0.1 mm;
inserting the catheter assembly into the first blood vessel;
advancing the catheter assembly along the first blood vessel while a distal portion of the first shaft extends distally beyond the distal end of the second shaft and while the guide wire is located in the first lumen in the first shaft and extends distally beyond the distal end of the first shaft;
advancing the catheter assembly from the first blood vessel to a second blood vessel having a branch that includes a third blood vessel; and
advancing at least the second catheter into the third blood vessel having a lesion on which catheter treatment is to be performed.

15. The method according to claim 14, further comprising removing the first shaft and the guide wire before advancing the second catheter into the third blood vessel.

16. The method according to claim 14, further comprising a rod fixed to the first catheter and extending in a proximal direction from the proximal end of the first catheter, the rod possessing a proximal end at which is fixed a hub.

17. The method according to claim 16, wherein the rod and the first shaft are other than coaxial.

18. The method according to claim 14, wherein the first catheter possesses a wall thickness T1, the second catheter possesses a wall thickness T2, and a clearance CL exists between an inner surface of the second shaft and an outer surface of the first shaft, and wherein T2<CL<T1.

19. The method according to claim 14, wherein the first catheter possesses a wall thickness T1, the second catheter possesses a wall thickness T2, and a clearance CL exists between an inner surface of the second shaft and an outer surface of the first shaft, and wherein T2<T1<CL.
